(19) United States
(12) Patent Application Publication
(10) Pub. No.: US 2021/0048434 A1
(43) Pub. Date: Feb. 18, 2021

(54) METHOD OF DETECTING BIO-MATERIAL

(71) Applicant: ELECTRONICS AND TELECOMMUNICATIONS RESEARCH INSTITUTE, Daejeon (KR)

(72) Inventors: Eun-ju JEONG, Daejeon (KR); Bong Kyu KIM, Daejeon (KR); Won Kyoung LEE, Daejeon (KR); CHUL HUH, Daejeon (KR)

(21) Appl. No.: 16/992,932

(22) Filed: Aug. 13, 2020

(30) Foreign Application Priority Data
Aug. 16, 2019 (KR) 10-2019-0100047

(51) Int. Cl. G01N 33/543; G01N 33/545

(52) U.S. Cl. CPC G01N 33/54346; G01N 33/545

(57) ABSTRACT

A method of detecting a bio-material is provided. According to exemplary embodiments of the inventive concept, the method of detecting a bio-material includes fixing a capture antibody to a surface of a fixing structure; providing a sensing antibody composite on the capture antibody; providing an analysis target sample including a bio-material between the capture antibody and the sensing antibody composite to form a composite layer; and providing a seed growing agent on the composite layer, wherein the sensing antibody composite includes an antigen binding part and a chain part, a gold nanoparticle is bonded to one terminal of the chain part, the composite layer includes the capture antibody, the bio-material bonded to the capture antibody, and the sensing antibody composite bonded to the bio-material, and the providing of the seed growing agent includes growing of a size of the gold nanoparticle.

### CROSS-REFERENCE TO RELATED APPLICATIONS

This U.S. non-provisional patent application claims priority under 35 U.S.C. § 119 of Korean Patent Application No. 10-2019-0100047, filed on Aug. 16, 2019, the entire contents of which are hereby incorporated by reference.

### BACKGROUND

The present disclosure herein relates to a method of detecting a bio-material, and more particularly, to a method of detecting a bio-material using gold nanoparticles.

An immune reaction is an antigen-antibody binding reaction by which an antigen and an antibody make selective binding and is widely used for bio-sensing the existence of a specific antigen or antibody in a specimen or for measuring the amount thereof. A typical immune reaction includes an enzyme immunoassay method which is a method of measuring the amount of an antigen-antibody reaction using enzyme as a predator. In this case, the enzyme predator may be replaced with a fluorescence material, a nanomaterial, a luminescence material, etc.

### SUMMARY

The present disclosure provides a method of detecting a bio-material by which detection time may be reduced.

The present disclosure also provides a method of detecting a bio-material, with improved detection efficiency.

The tasks for solving in the inventive concept is not limited to the above-described tasks, and unmentioned other tasks will be clearly understood by a person skilled in the art from the description below.

The present disclosure relates to a method of detecting a bio-material. According to exemplary embodiments of the inventive concept, the method of detecting a bio-material includes fixing a capture antibody to a surface of a fixing structure; providing a sensing antibody composite on the capture antibody; providing an analysis target sample including a bio-material between the capture antibody and the sensing antibody composite to form a composite layer; and providing a seed growing agent on the composite layer, wherein the sensing antibody composite includes an antigen binding part and a chain part, a gold nanoparticle is bonded to one terminal of the chain part, the composite layer includes the capture antibody, the bio-material bonded to the capture antibody, and the sensing antibody composite bonded to the bio-material, and the providing of the seed growing agent includes growing of a size of the gold nanoparticle.

In exemplary embodiments, the fixing of the capture antibody to the surface of the fixing structure may include providing a capture antibody dispersion in which the capture antibody is dispersed in a first solvent on the surface of the fixing structure; removing the first solvent; and first freeze-drying the fixing structure.

In exemplary embodiments, the providing of the sensing antibody composite on the capture antibody may include providing a sensing antibody composite dispersion in which the sensing antibody composite is dispersed in a second solvent on the surface of the fixing structure; removing the second solvent; and second freeze-drying the fixing structure.

In exemplary embodiments, the first freeze-drying and the second freeze-drying may be performed at a temperature of about −90° C. to about −70° C.

In exemplary embodiments, the fixing structure may include polystyrene.

In exemplary embodiments, the seed growing agent may include gold (Au) ions.

In exemplary embodiments, a diameter of the gold nanoparticle may be about 10 nm to about 50 nm.

In exemplary embodiments, the providing of the seed growing agent on the composite layer may include reducing the gold (Au) ions of the seed growing agent at a surface of the gold nanoparticle of the sensing antibody composite.

In exemplary embodiments, the bio-material may include Cardiac troponin I (cTnI), C-reactive protein (CRP), myoglobin, D-dimer, or creatine kinase (CK-MB).

In exemplary embodiments, the method may further include after providing the seed growing agent on the composite layer, applying light to the composite layer; and analyzing light passed through the composite layer to detect the bio-material, wherein the composite layer may include the capture antibody, the sensing antibody composite, and the bio-material.

In exemplary embodiments, the light applied to the composite layer may include light of a first wavelength, and the first wavelength may be about 450 nm to about 900 nm.

In exemplary embodiments, the gold nanoparticle of the sensing antibody composite in the composite layer may absorb light of a second wavelength among the light applied.

In exemplary embodiments, the second wavelength may be about 500 nm to about 510 nm.

In exemplary embodiments, the seed growing agent may include chloroauric acid (HAuCl₄) and hydroxylamine (NH₂OH).

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a flowchart of a method of detecting a bio-material according to exemplary embodiments of the inventive concept;

FIG. 2 is a schematic diagram showing a fixing structure where the detection reaction of a bio-material according to an embodiment takes place;

FIG. 3 is a schematic diagram showing a state where a capture antibody dispersion is provided in the reaction well of FIG. 2;

FIG. 4 is a schematic diagram showing a state where a capture antibody is fixed to the inner surface of the reaction well of FIG. 2;

FIG. 5 is a schematic diagram showing a state where a sensing antibody composite dispersion is provided in a reaction well;

FIG. 6 is a schematic diagram showing a state where a sensing antibody composite is fixed to the inside of a reaction well;

FIG. 7 is a schematic diagram showing a state where an analysis target sample is provided in the reaction well of FIG. 6;

FIG. 8 is a schematic diagram showing a state where a bio-material and a sensing antibody composite are bonded to a capture antibody;

FIG. 9 is a schematic diagram showing the inside of a reaction well after injecting a seed growing agent; and

FIG. 10 is a schematic diagram showing a light applying state to a composite layer.

### DETAILED DESCRIPTION

Hereinafter, preferred embodiments of the inventive concept will be described below in detail with reference to the accompanying drawings. The advantages and the features of the inventive concept, and methods for attaining them will be described in example embodiments below with reference to the accompanying drawings. The inventive concept may, however, be embodied in different forms and should not be construed as limited to the embodiments set forth herein. Rather, these embodiments are provided so that this description will be thorough and complete, and will fully convey the scope of the present inventive concept to those skilled in the art. The inventive concept is defined only by the scope of the claims.

The terminology used herein is for the purpose of describing exemplary embodiments only and is not intended to limit the present inventive concept. As used herein, the singular forms are intended to include the plural forms as well, unless the context clearly indicates otherwise. It will be further understood that the terms “comprises” and/or “comprising,” when used in this specification, specify the presence of stated features, steps, operations, and/or devices, but do not preclude the presence or addition of one or more other features, steps, operations, and/or devices thereof. In addition, reference symbols suggested according to the order of explanation should not be limited to their order, because the reference symbols are suggested in preferred embodiments. It will also be understood that when a layer is referred to as being ‘on’ another layer or substrate, it can be directly on the other layer or substrate, or third intervening layers may also be present.

It will be understood that, although the terms first, second, third etc. may be used herein to describe constituent elements (or structures), regions and layers should not be limited by these terms. These terms are only used to distinguish one constituent element (or structures) from another constituent elements (or structures). Thus, a first structure referred to in an embodiment could be termed a second structure in another embodiment. Example embodiments embodied and described herein may include complementary example embodiments thereof. Like reference numerals refer to like elements throughout.

The method of detecting a bio-material according to the inventive concept will be explained.

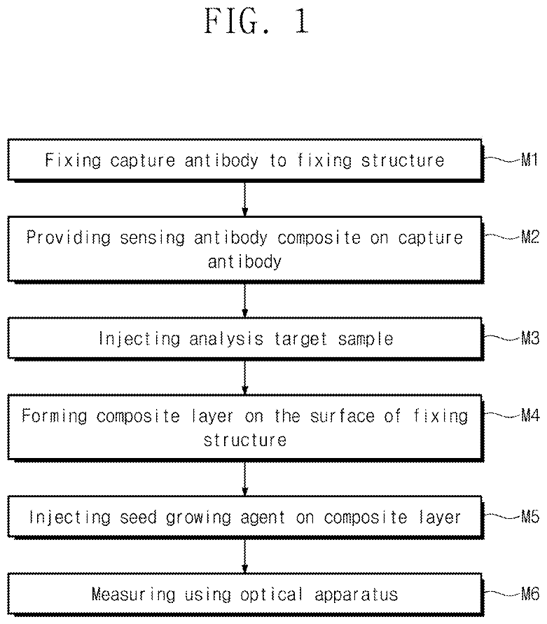

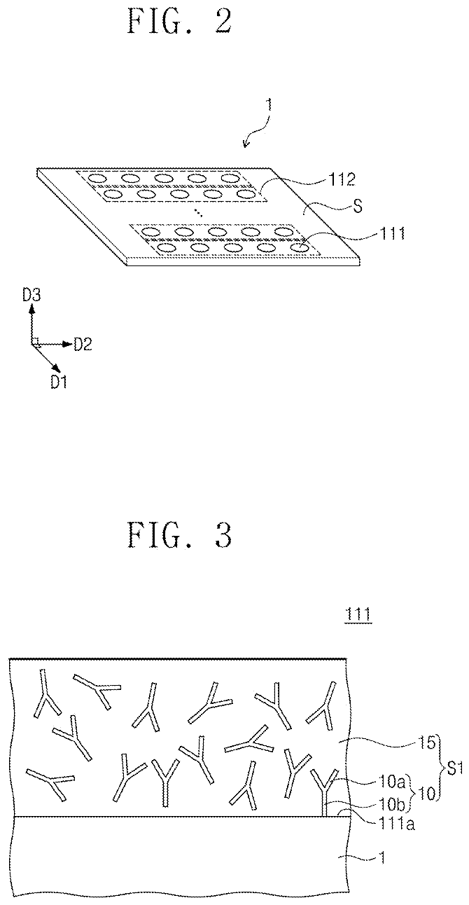

FIG. 1 is a flowchart of a method of detecting a bio-material according to exemplary embodiments of the inventive concept. FIG. 2 is a schematic diagram showing a fixing structure where the detection reaction of a bio-material according to an embodiment takes place.

Referring to FIG. 1 and FIG. 2, the method of detecting a bio-material according to an embodiment may include a step of fixing a capture antibody to a fixing structure (M1), a step of fixing a sensing antibody composite to the fixing structure (M2), a step of injecting an analysis target sample (M3), a step of injecting a seed growing agent (M4), and a step of measuring using an optical apparatus (M5).

In the step of fixing a capture antibody 10 to a fixing structure 1, a fixing structure 1 may be provided. The fixing structure 1 may provide a place where a capture antibody 10 and a sensing antibody composite 20, which will be explained later, are fixed and a bio-material detection reaction takes place. The fixing structure 1 may have, for example, a plate shape including a plurality of reaction wells 111. The reaction wells 111 of the fixing structure 1 may have a bowl shape in which a storing space is formed therein. The cross-sections of the reaction wells 111 may have circular shapes. However, an embodiment of the inventive concept is not limited thereto.

A first direction D1 may be in parallel to the top side S of the fixing structure 1. A second direction D2 may be in parallel to the top side S of the fixing structure 1 and in perpendicular to the first direction D1. A third direction D3 may be in perpendicular to the top side S of the fixing structure 1. The reaction wells 111 arranged in the second direction D2 may form one reaction well array 112. A plurality of reaction well arrays 112 may be separated in the first direction D1. The fixing structure 1 may include a polymer material. For example, the fixing structure 1 may include polystyrene PS. The fixing structure 1 may be transparent and transmit light. Meanwhile, different from the drawings, the fixing structure 1 may have a different shape. For example, the fixing structure 1 may be a tube having a capillary shape. Hereinafter, a single reaction well 111 will be explained.

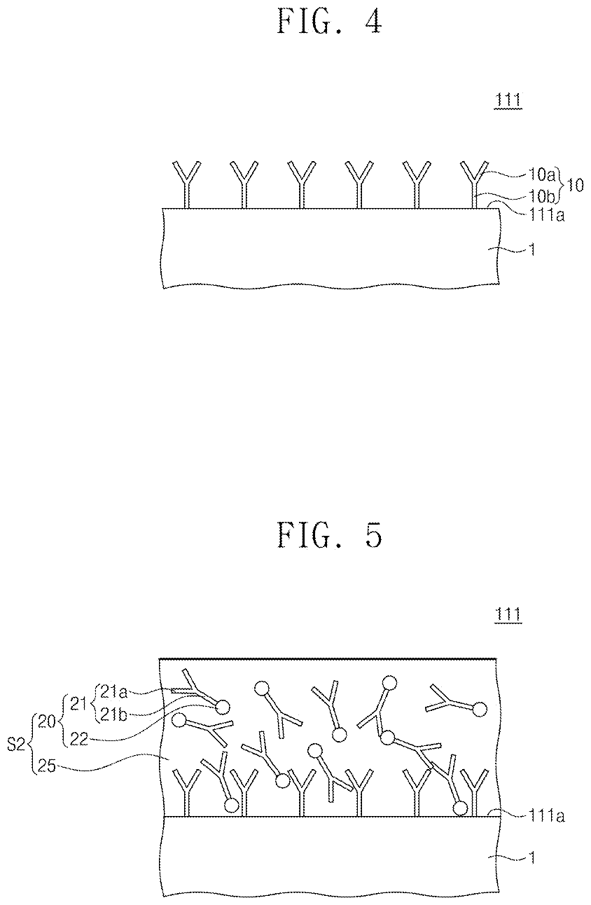

FIG. 3 is a schematic diagram showing a state where a capture antibody dispersion is provided in the reaction well of FIG. 2. FIG. 4 is a schematic diagram showing a state where a capture antibody is fixed to the inner surface of the reaction well of FIG. 2.

Referring to FIG. 3 and FIG. 4, in the step M1 of fixing the capture antibody 10 to the fixing structure 1, a capture antibody dispersion S1 may be provided in the reaction well 111 of the fixing structure 1. The capture antibody 10 may include an antigen binding part 10a and a chain part 10b. The antigen binding part 10a of the capture antibody 10 may make a specific bond with a bio-material 30 which will be explained later. In the capture antibody dispersion S1, a plurality of capture antibodies 10 may be dispersed in a first solvent 15. The first solvent 15 may include, for example, phosphate buffered saline (PBS). The fixing structure 1 may be stored in a refrigerator in conditions that the capture antibody dispersion S1 is provided in the reaction well 111. In this case, the temperature for cold storage may be about 3° C. to about 5° C. The cold storage may be kept for about 3 hours to about 5 hours. During the cold storage, the capture antibodies 10 of the capture antibody dispersion S1 may be fixed onto the inner surface 111a of the reaction well 111. As in FIG. 4, after finishing the cold storage, the first solvent 15 provided in the reaction well 111 may be removed. In this case, the capture antibodies 10 fixed to the inner surface 111a of the reaction well 111 may not be removed. The inside of the reaction well 111 may be washed using a washing solution. The washing solution may be, for example, a diluted solution of PBS. The washing process may be repeated several times. In this case, the capture antibodies 10 fixed to the inner surface 111a of the reaction well 111 may not be removed but remain on the surface 111a. After performing the washing process, the fixing structure 1 may be freeze-dried. The freeze-drying process may be performed at temperature conditions of about −90° C. to −70° C. The freeze-drying process may be performed under pressure conditions of about 80 mmHg. Through the freeze-drying process, the washing solution and the first solvent 15 remaining in the reaction well 111 may be removed. Accordingly, the capture antibodies 10 may be fixed even more firmly onto the inner surface 111a of the reaction well 111. The inner surface 111a of the reaction well 111 may be the same as the surface (now shown) of the fixing structure 1.

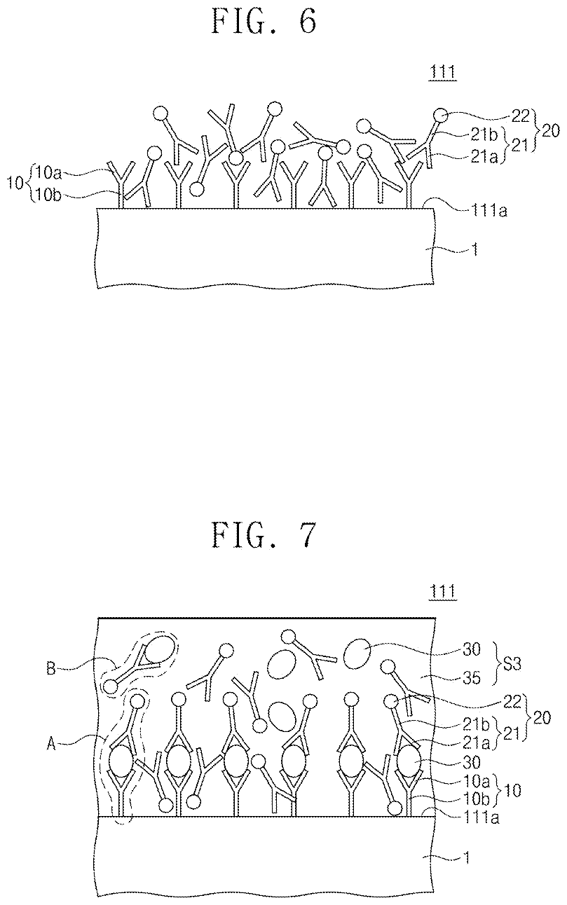

FIG. 5 is a schematic diagram showing a state where a sensing antibody composite dispersion is provided in a reaction well. FIG. 6 is a schematic diagram showing a state where a sensing antibody composite is fixed to the inside of a reaction well.

Referring to FIG. 5 and FIG. 6, in the step M2 of fixing the sensing antibody composite 20 to the fixing structure 1, a sensing antibody composite 20 may be prepared. The sensing antibody composite 20 may include a sensing antibody 21 and a gold nanoparticle 22. The sensing antibody 21 may include an antigen binding part 21a and a chain part 21b. The antigen binding part 21a of the sensing antibody composites 20 may make specific bonds with a bio-material which will be explained later. The gold nanoparticle 22 may be bonded to the terminal of the chain part 21b of the sensing antibody 21 to form the sensing antibody composite 20. The diameter of the gold nanoparticle 22 may be about 10 nm to about 30 nm.

A sensing antibody composite dispersion S2 may be provided in the reaction well 111 of the fixing structure 1. In the sensing antibody composite dispersion S2, sensing antibody composites 20 may be dispersed in a second solvent 25. The second solvent 25 may include, for example, PBS. The fixing structure 1 may be stored in a refrigerator in conditions that the sensing antibody composite dispersion S2 is provided in the reaction well 111. In this case, the temperature for cold storage may be about 3° C. to about 5° C. The cold storage may be kept for about 3 hours to about 5 hours. As shown in FIG. 6, during the cold storage, the sensing antibody composites 20 may be adsorbed onto the inner surface 111a of the reaction well 111 or onto the capture antibodies 10. After finishing the cold storage, the second solvent 25 provided in the reaction well 111 may be removed. In this case, the sensing antibody composites 20 fixed onto the capture antibodies 10 may not be removed. The inside of the reaction well 111 may be washed using a washing solution. The washing solution may be, for example, a diluted solution of PBS. The washing process may be repeated several times. In this case, the capture antibody composites 20 fixed onto the inner surface 111a of the reaction well 111 or onto the capture antibodies 10 may not be removed but remain. After performing the washing process, the fixing structure 1 may be freeze-dried. The freeze-drying process may be performed under temperature conditions of about −90° C. to about −70° C. The freeze-drying process may be performed under pressure conditions of about 80 mmHg. Through the freeze-drying process, the washing solution and the second solvent 25 remaining in the reaction well 111 may be removed.

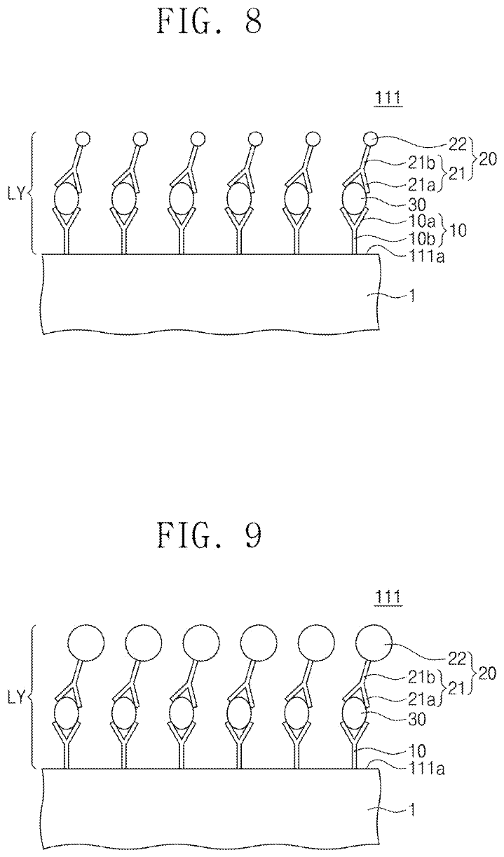

FIG. 7 is a schematic diagram showing a state where an analysis target sample is provided in the reaction well of FIG. 6. FIG. 8 is a schematic diagram showing a state where bio-materials and sensing antibody composites are combined with fixed antibodies.

Referring to FIG. 7, in the step M3 of injecting an analysis target sample S3 including a bio-material, an analysis target sample S3 may be prepared. The analysis target sample S3 may include bio-materials 30. For example, in the analysis target sample S3, bio-materials 30 may be dispersed in a solvent 35 in a liquid state. The bio-materials 30 may be an analysis target material. The bio-materials 30 may be obtained from the blood, urine, saliva, etc. of human and animals. The bio-materials 30 exist in a small quantity in the body and may be obtained in a small quantity. The bio-materials 30 may include nucleic acids, cells, viruses, proteins, and combinations thereof. If the bio-materials 30 are proteins, the bio-materials 30 may be any one among antigens, antibodies, matrix proteins, enzymes and coenzymes. The bio-materials 30 may play the role of a bio-marker for diagnosing diseases. For example, the bio-materials 30 may include Cardiac troponin I (cTnI), C-reactive protein (CRP), myoglobin, D-dimer, and/or creatine kinase (CK-MB) as a bio-marker for analyzing cardiovascular diseases.

Into the reaction well 111 of the fixing structure 1, the analysis target sample S3 may be injected. After injecting the analysis target sample S3 into the reaction well 111, the fixing structure 1 may be stood for a certain time period. The fixing structure 1 may be stood for about 4 minutes to about 6 minutes. Accordingly, a time period for combining the bio-materials 30, the capture antibodies 10, and the sensing antibody composites 20 may be provided. For example, the bio-materials 30 may make specific bonds with the capture antibodies 10. More particularly, the bio-materials 30 may make specific bonds with the antigen binding parts 10a of the capture antibodies 10. The antigen binding parts 10a of the capture antibodies 10 may be combined with the first parts of the bio-materials 30. The bio-materials 30 may be combined with the capture antibodies 10 and fixed onto the inner surface 111a of the reaction well 111 together. Accordingly, the bio-materials 30 may be exhibited on the inner surface 111a of the reaction well 111 (not shown). As in FIG. 7, the fixed sensing antibody composites 20 may have flowability due to the solvent 35. Any one among the sensing antibody composites 20 may be combined with any one among the bio-materials 30 fixed onto the inner surface 111a of the reaction well 111 (A of FIG. 7). More particularly, the bio-materials 30 may make specific bonds with the antigen binding parts 21a of the sensing antibody composites 20. The antigen binding parts 21a of the sensing antibody composites 20 may be combined with the second parts of the bio-materials 30. The second parts (not shown) of the bio-materials 30 may be different from the first parts (not shown) of the bio-materials 30. Accordingly, any one among the sensing antibody composites 20 may be fixed onto the inner surface 111a of the reaction well 111. Another one among the sensing antibody composites 20 may be combined with another one among the bio-materials 30 dispersed in the solvent 35 and having flowability (B of FIG. 7). In this case, another one among the sensing antibody composites 20 may not be fixed onto the inside of the reaction well 111.

After that, the solvent 35 in the reaction well 111 may be removed. In this case, the capture antibodies 10, the bio-materials 30, and/or the sensing antibody composites 20 fixed onto the inner surface 111a of the reaction well 111 may not be removed. The inside of the reaction well 111 may be washed using a washing solution. The washing solution may be, for example, a diluted solution of PBS. The washing process may be repeated several times. Unfixed composites of the sensing antibody composites 20 and the bio-materials 30 may be removed through the washing process.

Referring to FIG. 8, a composite layer LY may be formed on the inner surface 111a of the reaction well 111. The composite layer LY may include the capture antibodies 10, the bio-materials 30, and/or the sensing antibody composites 20 remaining after the washing process. The capture antibodies 10, the bio-materials 30, and/or the sensing antibody composites 20 may be combined with each other at the inner surface 111a of the reaction well 111 and fixed onto the inner surface 111a of the reaction well 111. The sensing antibody composites 20 may be combined with the bio-materials 30 and fixed onto the inner surface 111a of the reaction well 111. Since the antigen binding parts 21a of the sensing antibody composites 20 are combined with the bio-materials 30, the gold nanoparticles 22 of the sensing antibody composites 20 may be provided on the top part of the composite layer LY. More particularly, the gold nanoparticles 22 may be exposed to the top side of the composite layer LY and exposed to the outside. The gold nanoparticles 22 may be arranged toward the outside.

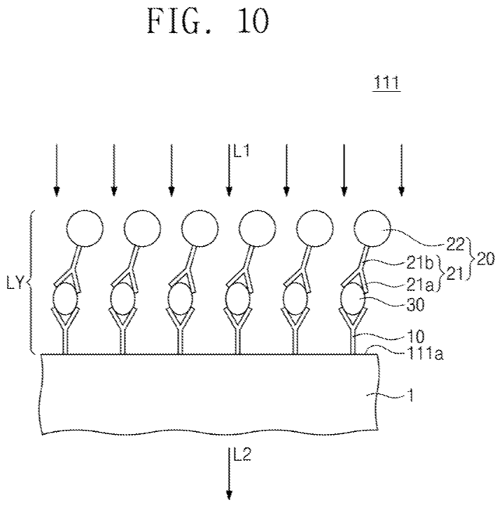

FIG. 9 is a schematic diagram showing the inside of a reaction well after injecting a seed growing agent. FIG. 10 is a schematic diagram showing a light applying state to a composite layer.

Referring to FIG. 1 and FIG. 9, in the step M4 of injecting a seed growing agent, a seed growing agent may be injected into the reaction well 111 (not shown). The seed growing agent may include chloroauric acid (HAuCl₄) and hydroxylamine (NH₂OH). More particularly, the seed growing agent may include gold ions (Au³⁺). The gold ions (Au³⁺) of the seed growing agent may be reduced at the surface of the gold nanoparticles 22 of the sensing antibody composites 20 and crystallized into gold (Au). Accordingly, the size of the gold nanoparticles 22 of the sensing antibody composites 20 may be enlarged.

Referring to FIG. 1 and FIG. 10, in the step M5 of measuring using an optical apparatus, light L1 may be applied in a perpendicular direction to the inner surface 111a of the reaction well 111. For example, the light L1 may be light having a first wavelength. The first wavelength may be about 450 nm to about 900 nm. A portion of the light L1 may pass through the composite layer LY and the fixing structure 1.

By analyzing the wavelength spectrum of the light L1 and light passed through L2, the bio-materials 30 in the analysis target sample S3 may be detected. The gold nanoparticles 22 of the sensing antibody composites 20 in the composite layer LY may absorb light of a second wavelength. For example, the second wavelength may be a wavelength of about 500 nm to about 510 nm. If the total volume of the gold nanoparticles 22 in the composite layer LY increases, the light passed through L2 may include little of the second wavelength.

The sensing antibody composites 20 are combined with the bio-materials 30, and if the amount of the bio-materials 30 combined with the capture antibodies 10 in the composite layer LY increases, a large amount of the gold nanoparticles 22 may be included in the composite layer LY. Accordingly, the detection sensitivity and accuracy of the bio-materials 30 may be improved.

In the method of detecting a bio-material according to an embodiment of the inventive concept, a free-drying process is performed, and the fixing structure 1 may be stored for a relatively long time period without damaging the capture antibodies 10 and the sensing antibody composites 20. For example, the storing time period may be about 4 months to about 6 months. Accordingly, the working process of fixing the capture antibodies 10 and the sensing antibody composites 20 onto the inner surface 111a of the fixing structure 1 may be omitted. If the bio-materials 30 are required to be detected and analyzed, the analysis target sample S3 may be immediately injected into the reaction well 111 of the fixing structure 1, thereby reducing detection time.

In the method of detecting a bio-material according to an embodiment of the inventive concept, a seed growing agent may be injected. If the amount of bio-materials 30 in an analysis target sample S3 is extremely small, the sensing antibody composites 20 in the composite layer LY may exist in an extremely small amount, and the amount of light L1 absorbed by the gold nanoparticles 22 of sensing antibody composites 20 may be very small. For example, the wavelength spectrum of light passed through L2 may be almost similar to the wavelength spectrum of light applied L1. The gold ions of the seed growing agent may enlarge the size of the gold nanoparticles 22 of the sensing antibody composites 20 existing in an extremely small amount in the composite layer LY. If the size of the gold nanoparticles 22 is enlarged, the light of the second wavelength may be absorbed even more. By analyzing the amount of the light absorbed, the existence of the bio-materials 30 in the analysis target sample may be effectively confirmed.

According to the method of detecting a bio-material according to exemplary embodiments of the inventive concept, the detection time of the bio-material to be detected may be reduced.

According to the method of detecting a bio-material according to exemplary embodiments of the inventive concept, the detection sensitivity of the bio-material to be detected may be improved.

The effects of the inventive concept are not limited to the above-described effects, and unmentioned other effects will be clearly understood by a person skilled in the art from the description above.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

What is claimed is:

1. A method of detecting a bio-material, the method comprising:
fixing a capture antibody to a surface of a fixing structure;
providing a sensing antibody composite on the capture antibody;
providing an analysis target sample comprising a bio-material between the capture antibody and the sensing antibody composite to form a composite layer; and
providing a seed growing agent on the composite layer,
wherein the sensing antibody composite comprises an antigen binding part and a chain part,
a gold nanoparticle is bonded to one terminal of the chain part,
the composite layer comprises the capture antibody, the bio-material bonded to the capture antibody, and the sensing antibody composite bonded to the bio-material, and
the providing of the seed growing agent comprises growing of a size of the gold nanoparticle.

2. The method of detecting a bio-material of claim 1, wherein the fixing of the capture antibody to the surface of the fixing structure comprises:
providing a capture antibody dispersion in which the capture antibody is dispersed in a first solvent on the surface of the fixing structure;
removing the first solvent; and
first freeze-drying the fixing structure.

3. The method of detecting a bio-material of claim 2, wherein the providing of the sensing antibody composite on the capture antibody comprises:
providing a sensing antibody composite dispersion in which the sensing antibody composite is dispersed in a second solvent on the surface of the fixing structure;
removing the second solvent; and
second freeze-drying the fixing structure.

4. The method of detecting a bio-material of claim 3, wherein the first freeze-drying and the second freeze-drying are performed at a temperature of about −90° C. to about −70° C.

5. The method of detecting a bio-material of claim 3, wherein the fixing structure comprises polystyrene.

6. The method of detecting a bio-material of claim 1, wherein the seed growing agent comprises gold (Au) ions.

7. The method of detecting a bio-material of claim 6, wherein a diameter of the gold nanoparticle is about 10 nm to about 50 nm.

8. The method of detecting a bio-material of claim 6, wherein the providing of the seed growing agent on the composite layer comprises:
reducing the gold (Au) ions of the seed growing agent at a surface of the gold nanoparticle of the sensing antibody composite.

9. The method of detecting a bio-material of claim 1, wherein the bio-material comprises Cardiac troponin I (cTnI), C-reactive protein (CRP), myoglobin, D-dimer, or creatine kinase (CK-MB).

10. The method of detecting a bio-material of claim 1, further comprising after providing the seed growing agent on the composite layer:
applying light to the composite layer; and
analyzing light passed through the composite layer to detect the bio-material.

11. The method of detecting a bio-material of claim 10, wherein the light applied to the composite layer comprises light of a first wavelength, and
the first wavelength is about 450 nm to about 900 nm.

12. The method of detecting a bio-material of claim 11, wherein the gold nanoparticle of the sensing antibody composite in the composite layer absorbs light of a second wavelength among the light applied.

13. The method of detecting a bio-material of claim 12, wherein the second wavelength is about 500 nm to about 510 nm.

14. The method of detecting a bio-material of claim 1, wherein the seed growing agent comprises chloroauric acid (HAuCl₄) and hydroxylamine (NH₂OH).
